(19) United States
(12) Patent Application Publication
(10) Pub. No.: US 2007/0281926 A1
(43) Pub. Date: Dec. 6, 2007

(54) Rectal Composition

(76) Inventors: Anders Carlsson, Stockholm (SE); Bengt Herslof, Stockholm (SE); Conny Bogentoft, Hasselby (SE)

Correspondence Address: DICKSTEIN SHAPIRO LLP, 1177 AVENUE OF THE AMERICAS (6TH AVENUE), NEW YORK, NY, 10036-2714, US

(21) Appl. No.: 10/597,714

(22) PCT Filed: Feb. 4, 2005
(86) PCT No.: PCT/SE05/00139

§ 371 Date: Apr. 27, 2007

(30) Foreign Application Priority Data
Feb. 4, 2004 (SE) 0400233-3

(51) Int. Cl. A61K 47/12; A61K 31/135; A61K 31/19; A61K 31/195; A61K 31/35; A61K 31/415; A61P 1/10; A61K 31/44; A61K 31/445; A61K 31/54; A61K 31/55

(52) U.S. Cl. 514/221; 514/226.2; 514/226.5; 514/282; 514/322; 514/353; 514/398; 514/563; 514/567; 514/568; 514/569; 514/653; 514/786

(57) ABSTRACT

A viscous or gellous pharmaceutical composition for the treatment of constipation by rectal administration comprises a polar lipid component, a polyvalent alcohol component, water, and optionally an oily triglyceride. Also disclosed is a compressible device filled with a single dose of the composition and a method for its manufacture; a method of treating constipation, comprising rectal administration of a constipation-dissolving amount of the composition; and a method of manufacture of a medicament for treating constipation comprising the composition of the invention.

### FIELD OF THE INVENTION

The present invention relates to a composition for rectal administration for the treatment of constipation, a method for its preparation, and its use.

### BACKGROUND OF THE INVENTION

Constipation is often defined as a frequency of defecation of twice per week or less but frequency is not the only sufficient criterion. Most individuals who describe them as constipated complain of excessive straining or discomfort at defecation or passage of hard or pellet stools, although the frequency of defecation is within the normal range (A Wald, Constipation. Adv Gastroenterol 2000;84 (5) 1231-1246).

Constipation is a serious problem affecting many people. In the United States about a fourth of elderly men and a third of elderly women are affected (D C Schaeffer and L J Cheskin, Constipation in the Elderly. Am Fam Physician 1998; 58 (4) 907-914). At least 75 percent of elderly hospitalized patients and nursing home residents use laxatives for bowel regulation (W R Primrose et al., Prescribing patterns observed in registered nursing homes and long-stay geriatric wards. Age Ageing 1987; 16: 25-28).

While a diet rich in natural fiber and physical activity may alleviate and even prevent constipation, this is not true or possible for various reasons for a large number of affected persons. The use of laxatives thus is the remedy most often relied on to fight constipation. They are however not free of drawbacks, such as a substantial delay between administration and onset of effect or irritation of the bowel when used over a long period of time. Another way to treat constipation is by enemas. A drawback with enemas is that their administration is problematic for reasons of leakage.

### OBJECTS OF THE INVENTION

It is an object of the present invention to provide a means for treating constipation that is efficient and has a rapid onset of action, does not irritate the mucus of the bowel, and is convenient to administer.

Further objects of the invention will be understood from the following description of the invention and preferred embodiments thereof as well as from the appended claims.

### SUMMARY OF THE INVENTION

According to the present invention is disclosed a pharmaceutical composition for the treatment of constipation by rectal administration comprising a polar lipid component, an oily triglyceride component, a polyvalent alcohol component, and water.

It is preferred for the triglyceride oil component to be a fraction of natural triglyceride, in particular a vegetable oil. The term “oily triglyceride component” relates to triglyceride of oily consistence at a temperature of 20° C. The term triglyceride includes mixtures of triglycerides.

It is preferred for the polar lipid component to consist of polar lipid, the polar lipid being preferably galactolipid, even more preferred digalactosyldiacylglycerol. In this application the term polar lipid comprises a mixture of polar lipids; the term galactolipid comprises a mixture of galactolipids.

It is preferred for the polyvalent alcohol component to comprise one or more of glycerol, propylene glycol, butylene glycol, pentylene glycol, hexylene glycol, butylene-1,4-diol, pentylene-1,5-diol, hexylene-1,6-diol, and macrogol. Particularly preferred are glycerol and propylene glycol. Most preferred is glycerol.

It is particularly preferred for the pharmaceutical composition of the invention to comprise from 0 percent to 30 percent of oily triglyceride, from 0.5 to 30 percent of polar lipid component, more preferred from 5 percent to about 25 percent, most preferred from about 10 percent to about 20 percent.

According to a first preferred aspect the pharmaceutical composition of the invention is of a creamy consistence and comprises from 5 to 30 percent of oily triglyceride.

According to a second preferred aspect the pharmaceutical composition of the invention is of a gellous or viscous consistence and comprises from 5 percent to 30 percent of polar lipid component, more preferred from 8 percent to about 25 percent, most preferred from about 10 percent to about 20 percent, while it is free from oily triglyceride.

According to a third preferred aspect the pharmaceutical composition of the invention is of a creamy consistence and comprises from 1 percent to 20 percent of fractionated oat oil component, more preferred from 2 percent to 15 percent, most preferred from 4 percent to 10 percent.

According to a fourth preferred aspect the pharmaceutical composition of the invention comprises from 5 percent to 75 percent of polyvalent alcohol component, more preferred from 8 percent to 70 percent, most preferred from about 10 percent to about 70 percent.

According to a fifth preferred aspect the pharmaceutical composition of the invention essentially consists of from 8 to 25 percent of galactolipid, from 8 to 75 percent of glycerol, and from 20 to 75 percent of water, with the proviso that said components add up to 100 percent.

According to a sixth preferred aspect the pharmaceutical composition of the invention has a dynamic viscosity at 20° C. of at least y·10⁻³ Ns/m², y being 2.5 or more, preferably about 4 more, more preferred about 9 or more, most preferred about 30 or more.

The composition of the invention may additionally contain one or more of colourant, preservative, fragrance, UV-stabilizing agent, antioxidant or similar.

According to the invention is also disclosed a device, such as a disposable syringe, filled with a single dose of the composition of the invention. The amount of composition in the device may vary within wide limits but will preferably be from 5 to 30 ml, more preferred from 10 to 20 ml. It is also possible to provide the single dose in a soft polymer bag provided with a sealed mouthpiece, which is removed prior to use, from which it can be squeezed out for rectal administration. The invention also comprises a method of manufacture of the device, comprising providing the composition of the invention, providing a compressible container with a mouthpiece suited for rectal administration, filling the container with a single dose of the composition of the invention, and sealing the container and/or the mouthpiece. It is understood that the space in the container filled with the composition and the mouthpiece are in communication. The mouthpiece tip must be sealed either before or after filling with a seal that can be removed prior to administration. The seal may also have the form of a breakable mouthpiece tip provided by an indication of fracture. For rectal administration of the composition of the invention also conventional plunger type, positive-displacement syringes with a short and wide nozzle may be used. A nozzle diameter of, for instance, 12 mm and more is suitable. The nozzle is provided with a bulbous end to which a flexible polymer tube of corresponding diameter and a non-critical length of about 20 cm is connected for insertion into the rectum. Also disclosed is the aforementioned compressible device filled with a single dose of the composition of the invention. According to the invention is also disclosed a method of manufacture of the aforementioned device, comprising providing the composition of the invention, providing a compressible container with a mouthpiece suited for rectal administration, filling the container with a single dose of the composition, and sealing the container and/or the mouthpiece.

A single dose of the composition of the invention can be administered by rectal injection within a rather short period of time, such as from 5 to 60 seconds.

The composition of the invention of a gellous consistence or the consistence of highly viscous liquid, which is free from oily triglyceride, can be prepared by mixing the polar lipid component and the polyvalent alcohol component, followed by the addition of water mixing. Air bubbles enclosed in the composition can be removed by centrifugation. The composition is allowed to stand for a selected period of time, such as 12 hours or more, to complete salvation (swelling) of the polar lipid component. Swelling is facilitated by a short treatment with a high-shear mixer or similar high-shear agitation.

The composition of the invention of a creamy consistence comprising oily triglyceride can be prepared by separately mixing the galactolipid component, in particular fractionated oat oil component, and the oily triglyceride component, in particular of vegetable origin, at the one hand, and the polyvalent alcohol component and water, at the other hand. The thus formed oil and aqueous phases are heated, such as to a temperature of about 65° C. to 70° C. The warm oil phase is then poured into the warm aqueous phase while mixing at a high shear rate. The thus formed pre-emulsion is further homogenized in a warm state. After cooling to room temperature, the composition has the form of a smooth, viscous cream.

According to a seventh preferred aspect of the invention is disclosed a method of treating constipation, the method comprising rectal administration of a constipation-dissolving amount, such as from 5 to 50 ml, of the composition of the invention to a person suffering from constipation.

According to an eight preferred aspect of the invention is disclosed the therapeutic use of the composition of the invention, in particular the use for treating constipation.

According to a ninth preferred aspect of the invention is disclosed a method for the manufacture of a medicament for treating constipation, the method comprising blending a polar lipid component, a polyvalent alcohol component, water and, optionally, an oily triglyceride, to form a gellous or viscous solution.

According to a tenth preferred aspect a pharmacologically active agent can be incorporated in the composition of the invention by dissolution or suspension. Particularly preferred for such incorporation are agents that are known to be administered per rectum, such as sulphasalazine, 5-amino-salicylate, sodium aminosalicylate, diazepam, chlorpromazine, tramadol, morphine, domperidone, piroxicam, paracetamol, indomethacin, diclofenac, naproxen, metronidazole, antibiotics and antimycotics but also local anaesthetics for use in hemorrhoid treatment such as lidocaine. The thus modified composition can be used for rectal administration of the pharmacologically active agent.

According to the invention is also disclosed the use of the composition of the invention for treating constipation.

Also disclosed is the use of the composition for rectal administration of a pharmacologically active agent. Furthermore, according to the invention, is disclosed a method of manufacture of a medicament for treating constipation, comprising blending a polar lipid component, a polyvalent alcohol component, water and, optionally, an oily triglyceride, to form a gellous or viscous solution.

The composition of the invention is remarkably physically stable. Depending on its composition its consistence may be that of a cream, a gel or a highly viscous liquid. Its consistence or viscosity is only moderately affected by a change in temperature; for example, it can be transferred directly from the refrigerator (at 4° C.) to a syringe for administration and administered to a patient at that temperature.

The invention will now be explained in greater detail by reference to preferred but not limiting embodiments thereof.

### DESCRIPTION OF PREFERRED EMBODIMENTS

### EXAMPLE 1

General method of preparation of the composition of the invention of gellous consistence or of the consistence of a viscous liquid. 20.0 g of galactolipid (CPL®-Galactolipid; Lipid Technologies Provider AB, Karlshamn, Sweden) and 20.0 g of glycerol were mixed by hand in a plastic container and allowed to stand for 30 min. Water (60 ml) was added and the contents were again mixed by hand and left over night at room temperature. After repeating the mixing by hand the mixture was centrifuged 10 min at 1500 rpm using a Jouan bench centrifuge to remove air bubbles. Mixing and centrifugation was repeated once. The resulting formulation was a clear viscous gel, which can be stored at room temperature or in a refrigerator. By lowering the amount of galactolipid below about eighth percent by weight, a viscous liquid is obtained; it is prepared in the same manner as described above.

### EXAMPLE 2a

General method A of preparation of the composition of the invention of a creamy consistence. Fractionated oat oil (10 g; Lipid Technologies Provider AB, Karlshamn, Sweden) and corn oil (10 g) were mixed in a beaker and then stirred with a magnetic stirrer for 30 min when the fractionated oil component had dispersed completely to form an oil phase. Glycerol (40 g) and water (40 ml) were mixed in a second beaker to form an aqueous phase. The oil and aqueous phases were heated to 65° C. to 70° C., and the warm oil phase was poured into the warm aqueous phase during high-shear mixing (Polytron PT-MR 3000). After the end of addition mixing was continued for 2 min at 15,000 rpm. The pre-emulsion thus formed was homogenized twice at 200 psi in an ultrasonic homogeniser (Branson Minisonic 4). The product was allowed to cool in a water bath. It had the form of a smooth viscous cream.

### EXAMPLE 2b

General method B of preparation of the composition of the invention of a creamy consistence. Fractionated oat oil (3 g; Lipid Technologies Provider AB, Karlshamn, Sweden), cetostearyl alcohol (6 g; Lanette 0; Cognis Corp., Hoboken, N.J.), glyceryl stearate (3 g; Admul M G; Quest International, Ashford, Kent, UK) and rapeseed oil (15 g) were weighed in a beaker and melted at about 65° C. and then stirred with a spatula by hand for a few minutes until the components had mixed completely to form an oil phase. Glycerol (30 g) and water (93 ml) were mixed in a second beaker to form an aqueous phase. The oil and aqueous phases were heated to 65° C. and 45° C., respectively, and the warm oil phase was poured into the warm aqueous phase during mixing with a spatula. The mixture was then subjected to high-shear mixing (Ultra-Turrax) at approximately 8,000 rpm for 30 seconds. The emulsion thus formed was transferred to a plastic container with a cover and the product was allowed to cool at room temperature. It had the form of a smooth viscous cream.

### EXAMPLE 2c

General method C of preparation of the composition of the invention of a creamy consistence. CPL®-Galactolipid (1.5 g; Lipid Technologies Provider AB, Karlshamn, Sweden), CPL®-Evening Primrose oil (20 g; Lipid Technologies Provider AB, Karlshamn, Sweden), and ascorbyl palmitate (0.02 g) were mixed in a beaker and then stirred with a magnetic stirrer until the galactolipid was completely dispersed, that is, for 30-60 min. Glycerol (10 g), methyl-p-hydroxybenzoate (0.40 g), propyl-p-hydroxybenzoate (0.24 g) and purified water (58.84 g) were mixed in a second beaker while stirring to form an aqueous phase. When the oil phase had a homogeneous appearance, cetostearyl alcohol (7 g) and glyceryl stearate (2 g) was added. The oil phase and the aqueous phase were both heated to 55° C. while stirring. The warm oil phase was added to the warm aqueous phase during high-shear mixing (Polytron PT-MR 3000). After addition of the oil phase the pre-emulsification continued for 2 min at 15,000 rpm. The pre-emulsion was then homogenised 6 times at 200 psi in an Ultrasonic homogeniser (Branson Minisonic 4). The cream was allowed to cool in a water bath.

### EXAMPLE 3

Preparation of compositions of the invention. A number of compositions according to the invention listed in Table 1 were prepared by the general methods of Examples 1 and 2 in varying batch sizes. A gellous composition (A) for rectal administration which is not a composition of the invention is also shown.

TABLE 1
Compositions of the invention (Nos. 1-27)

| | Components (percent by weight) | | | Batch | |
|---|---|---|---|---|---|
| Comp. | | Glycerol or | | size | |
| No. | Galactolipid | PG**) or BD***) | Water | (g) | Appearance |
| 1 | 20 | 55 | 25 | 343 | Clear yellow-brown gel |
| 2 | 20 | 50 | 30 | 200 | Clear yellow-brown gel |
| 3 | 20 | 50 | 30 | 50 | Clear yellow-brown gel |
| 4 | 20 | 30 | 50 | 50 | Clear yellow-brown gel |
| 5 | 15 | 50 | 35 | 50 | Clear yellow-brown gel |
| 6 | 15 | 35 | 50 | 50 | Clear yellow-brown gel |
| 7 | 10 | 50 | 40 | 100 | Clear yellow-brown gel |
| 8 | 15 | 53 | 32 | 100 | Clear yellow-brown gel |
| 9 | 10 | 50 | 40 | 100 | Clear yellow-brown gel |
| 10 | 15 | 45 | 40 | 100 | Clear yellow-brown gel |
| 11 | 10 | 50 | 40 | 100 | Clear yellow-brown gel |
| 12 | 20 | 30 | 50 | 100 | Clear yellow-brown gel |
| 13 | 20 | 10 | 70 | 100 | Clear yellow-brown gel |
| 14 | 20 | 20 | 60 | 100 | Clear yellow-brown gel |
| 15 | 20 | 30 | 50 | 100 | Clear yellow-brown gel |
| 16 | 20 | 40 | 40 | 100 | Clear yellow-brown gel |
| 17 | 20 | 55 | 25 | 100 | Clear yellow-brown gel |
| 18 | 10 | 40 | 40 | 100 | +10% FOO; yellow-brown cream |
| 19 | 5 | 55 | 40 | 100 | Opaque yellow-brown viscous liquid |
| 20 | 5 | 25 | 70 | 100 | Slightly milky yellow-brown viscous liquid |
| 21 | 5 | 55 | 40 | 100 | Opaque yellow-brown viscous liquid |
| 22 | 5 | 75 | 20 | 100 | Nearly clear yellow-brown viscous liquid |
| 23 | 5 | 5 | 90 | 100 | Milky yellow-brown viscous liquid |
| 24 | 10 | 20 PG | 70 | 50 | Semi-clear yellow-brown viscous liquid |
| 25 | 18 | 30 PG | 52 | 50 | Semi-clear yellow-brown viscous liquid |
| 26 | 20 | 30 BD | 50 | 50 | Milky yellow-brown viscous liquid |
| 27 | 10 | 10 BD | 80 | 50 | Semi-clear yellow-brown viscous liquid |
| A*) | — | — | 89 | 100 | Milky yellow-brown gel |

*)Comprises additionally 10 percent of fractionated oat oil (FOO; non-polar triglyceride oil) and 1 percent of Carbopol 974P, a cross-linked polyacrylic acid marketed by Noveon Inc., Cleveland, Ohio, USA)
**)Propylene glycol
***)1,3-Butanediol

TABLE 2
Compositions of the invention (Nos. 28-32)

| | | | | | | Batch | |
|---|---|---|---|---|---|---|---|
| Comp. | Components (percent by weight) | | | | | size | |
| No. | FOO | TG | Glycerol | Thick.*) | Water | (g) | Appearance |
| 28 | 2 | 10 | 20 | 6 | 62 | 150 | Milky yellow-brown cream |
| 29 | 2 | 10 | 30 | 6 | 52 | 150 | Milky yellow-brown cream |
| 30 | 2 | 10 | 40 | 6 | 42 | 150 | Milky yellow-brown cream |
| 31 | 2 | 10 | 50 | 6 | 32 | 150 | Milky yellow-brown cream |
| 32 | 2 | 10 | 30 | 3 | 55 | 150 | Milky yellow-brown cream |

*)Thickeners: cetostearyl alcohol (Admul MG) plus glyceryl stearate (Lanette O)

### EXAMPLE 4

Comparative Test 1 of Constipation-Releasing Effect.

Composition no. 1 (Table 1), which is a composition of the invention, was compared with composition A (Table 1), which is a gellous composition of similar physical appearance but substantially different from and thus not comprised by the composition of the invention. In this test three healthy persons compared the aforementioned gellous compositions in regard of their efficiency to trigger the need for rectal emptying. The results are shown in Table 3. In assessing the various variables the test persons used a scale from 0 to 10 where 0 signifies best and 10 worst in regard of volume, irritation and viscosity of/caused by the respective preparation, and where 0 signifies worst and 10 best in regard of effect. The test was carried out in the following manner. The tip of the syringe containing 10 g of the preparation was inserted into the rectum until a stop was felt. Then the sample was injected. The test person was told to stand up and walk around for one min and then to sit down for 15 min. After an interval of two hours the test person carried out the same procedure with the other formulation.

TABLE 3
Comparison of constipation-releasing effect

| | | | | | | | Difficulty |
|---|---|---|---|---|---|---|---|
| | | | | | Time from | | of |
| | | | Irritation | Viscosity | admini- | | keeping |
| | | Volume | caused by | of | stration to | | composi- |
| Composi- | Test | of composi- | composi- | composi- | toilet visit | | tion for |
| tion | person | tion | tion | tion | (min) | Effect | 15 min |
| 1 | 1 | 0 | 0 | 0 | 5 | 10 | yes |
| | 2 | 0 | 0 | 0 | 5 | 10 | yes |
| | 3 | 0 | 0 | 0 | 5 | 10 | yes |
| A | 1 | 0 | 0 | 0 | 15 | 5 | no |
| | 2 | 0 | 0 | 0 | 60 | 4 | no |
| | 3 | 0 | 0 | 0 | 15 | 3 | no |

Comparative Test 2 of Constipation-Releasing Effect.

Compositions no. 28-32 (Table 2), which are compositions of the invention, were compared by this test. In two adult healthy persons the compositions of creamy consistency were compared in regard of their efficiency to trigger the need for rectal emptying. The results are shown in Table 4. In assessing the various variables the test persons used a scale from 0 to 10 where 0 signifies best and 10 worst in regard of volume, irritation and viscosity of/caused by the respective preparation, and where 0 signifies worst and 10 best in regard of effect. The test was carried out in the same manner as in Comparative test 1.

TABLE 4
Comparison of constipation-releasing effect

| | | | Irritation | | Time from | | Difficulty of |
|---|---|---|---|---|---|---|---|
| | Test | Volume of | caused by | Viscosity of | administration to | | keeping composi- |
| Composition | person | composition | composition | composition | toilet visit (min) | Effect | tion for 15 min |
| 28 | 4 | 0 | 0 | 0 | 36 | 2 | no |
| | 5 | 3 | 2 | 1 | 10 | 8 | yes |
| 29 | 4 | 0 | 0 | 0 | 9 | 9 | no |
| | 5 | 4 | 0 | 1 | 13 | 8 | yes |
| 30 | 4 | 0 | 0 | 0 | 25 | 4 | no |
| | 5 | 3 | 1 | 3 | 18 | 8 | no |
| 31 | 4 | 0 | 0 | 0 | 23 | 4 | no |
| | 5 | 5 | 0 | 1 | 20 | 8 | no |
| 32 | 4 | 0 | 0 | 0 | 4, 16 | 10 | yes |
| | 5 | 7 | 1 | 1 | 60 | 3 | no |

### EXAMPLE 5

Preparation of the Composition of the Invention Comprising a Pharmacologically Active Agent

Lidocaine composition A. 0.20 g of lidocaine hydrochloride (Sigma-Aldrich, L5647) was added to 9.80 g of composition no. 16 (Table 1) and mixed gently by hand. The resulting composition was a milky yellow-brown viscous liquid.

Lidocaine composition B. 0.03 g of lidocaine hydrochloride (Sigma-Aldrich, L5647) was added to 2.83 g of composition no. 28 (Table 2). The mixture was melted and mixed gently by hand. The resulting composition was a milky yellow-brown viscous cream.

5-Aminosalicylic acid composition A. 0.50 g of powderous 5-aminosalicylic acid, 95% (Sigma-Aldrich, A79809) was suspended in 37.0 g of warm (50° C.) water using a magnetic stirrer. 3.0 g of glycerol was added to the suspension, followed by the addition of 10.0 g of galactolipid in two portions. The mixture was stirred using a spatula and was then left over night at room temperature (21° C.). The resulting composition was a brown highly viscous suspension.

5-Aminosalicylic acid composition B. 0.033 g of powderous 5-aminosalicylic acid, 95% (Sigma-Aldrich, A79809) was suspended in 0.35 g of warm (50° C.) rapeseed oil and mixed by hand. 3.27 g of composition no. 28 (Table 2) was added to the suspension. The mixture was heated (50° C.) and mixed by hand. The resulting composition was a yellow-brown viscous cream.

All compositions were easily administered rectally from a syringe.

### EXAMPLE 6

Viscosity Measurements

The dynamic viscosity at 20° C. of compositions no. 20 and no. 23 (Table 1) representing compositions near the low end of the useful viscosity range was estimated in the following way. A 5 ml volume pipette was clamped in an upright position and filled with sample up to the volume mark, and was then allowed to drain. The time for draining to a mark 10 cm below the volume mark was recorded. Pure water was used as a reference.

It was assumed that the tested compositions of the invention behaved as Newtonian fluids. Their viscosity was calculated using the equation ηₛₐₘₚₗₑ=η_{water}·ρₛₐₘₚₗₑ/ρ_{water}·tₛₐₘₚₗₑ/t_{water}. The densities for 5% and 25% by weight of glycerol in water at 20° C., 1.010 and 1.059 kg/dm³ calculation (Handbook of Chemistry and Physics, 60^{th} Ed.), respectively, were substituted in the equation for the unknown density of compositions no. 23 and 20, respectively. The other constants used were ρ_{water}=0.998 kg/dm³ and η_{water}=1.00·10−3 Ns/m² (1 cP).

The following tₛₐₘₚₗₑ values were recorded at 20° C.: water, 5 s; composition no. 23, 19 s; composition no. 20, 42 s. The dynamic viscosity of composition 20, containing 5% of galactolipid and 25% of glycerol, was calculated to be 8.9·10⁻³ Ns/m², and that of composition no. 23, containing 5% galactolipid and 5% glycerol, to be 3.8·10⁻³ Ns/m².

1. Pharmaceutical composition for the treatment of constipation by rectal administration comprising a polar lipid component, a polyvalent alcohol component, water, and optionally an oily triglyceride.

2. The composition of claim 1, wherein the oily triglyceride component is present and is a fraction of natural triglyceride.

3. The composition of claim 2, wherein the oily triglyceride component comprises fractionated oat oil.

4. The composition of claim 1, wherein the polar lipid component comprises galactolipid.

5. The composition of claim 4, wherein the galactolipid comprises digalactosyldiacylglycerol.

6. The composition of claim 1, wherein the polyvalent alcohol component is selected from the group consisting of glycerol, propylene glycol, butylene glycol, pentylene glycol, hexylene glycol, butylene-1,4-diol, pentylene-1,5-diol, hexylene-1,6-diol, macrogol, and mixtures thereof.

7. The composition of claim 6, wherein the polyvalent alcohol component comprises glycerol or propylene glycol.

8. (canceled)

9. The composition of claim 1, comprising from 0 percent to 30 percent of oily triglyceride and from 0.5 to 30 percent of polar lipid component.

10. The composition of claim 1 of a creamy consistence, comprising from 5 to 30 percent of oily triglyceride.

11. The composition of claim 1 of a gellous or viscous consistence, comprising from 5 percent to 30 percent of polar lipid component, with the provisio that it is free from oily triglyceride.

12. The composition of claim 11, comprising from 8 percent to 25 percent of polar lipid component.

13. (canceled)

14. The composition of claim 11, comprising from 5 percent to 75 percent of polyvalent alcohol component.

15. The composition of claim 11, comprising from 8 percent to 70 percent of polyvalent alcohol component and from 10 percent to 20 percent of polar lipid component.

16. (canceled)

17. The composition of claim 11, essentially consisting of from 8 to 25 percent of galactolipid, from 8 to 75 percent of glycerol, and from 20 to 75 percent of water, with the proviso that said components add up to 100 percent.

18. The composition of from 10 percent to 20 percent of polar lipid component comprising a pharmacologically active agent.

19. The composition of claim 18, wherein the pharmacologically active agent is selected from the group consisting of sulphasalazine, sodium aminosalicylate, diazepam, chlorpromazine, tramadol, morphine, domperidone, piroxicam, paracetamol, indomethacin, diclofenac, naproxen, metronidazole, antibiotics, antimycotics, and hemorrhoid treatment local anaesthetics.

20. (canceled)

21. The composition of claim 1 having a dynamic viscosity at 20° C. of at least 2.5·10⁻³ Ns/m².

22. The composition of claim 21 having a dynamic viscosity at 20° C. of at least about 5·10⁻³ Ns/m².

23. The composition of claim 21 having a dynamic viscosity at 20° C. of at least about 9·10⁻³ Ns/m².

24. Compressible device filled with a single dose of the composition of claim 1 and having with a sealed mouthpiece adapted for rectal administration.

25. The device of claim 24 in form of a syringe or soft polymer container.

26. (canceled)

27. The device of claim 24, wherein the single dose has a volume of from 5 to 50 ml.

28. A method of treating constipation, comprising rectal administration of a constipation-dissolving amount of the composition of claim 1 to a person suffering from constipation.

29-32. (canceled)
